(19) United States
(12) Patent Application Publication
(10) Pub. No.: US 2010/0318063 A1
(43) Pub. Date: Dec. 16, 2010

(54) DUAL BARREL SYRINGE ASSEMBLY

(75) Inventor: David B. SOLL, Ambler, PA (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: CLEO COSMETIC AND PHARMACEUTICALS CO., LLC, Ambler, PA (US)

(21) Appl. No.: 12/797,676

(22) Filed: Jun. 10, 2010

Related U.S. Application Data
(60) Provisional application No. 61/185,828, filed on Jun. 10, 2009.

(51) Int. Cl. A61M 5/31; A61J 1/14

(52) U.S. Cl. 604/518; 604/416; 604/82

(57) ABSTRACT

A syringe system for dispensing or injecting one or more fluids is provided. The syringe system includes a hollow body that retains cartridge(s) and/or syringe(s) each containing a fluid, such as an injectable fluid e.g., a catalyst in one cartridge and a polymer in the other. Upon use for dispensing two fluids, the syringe system mixes the two fluids immediately prior to or concurrently with the dispensing or injecting of the fluids. The syringe system can also be configured to independently dispense a single fluid or immediately dispense multiple fluids sequentially.

### CROSS-REFERENCE TO RELATED APPLICATIONS

This application is entitled to and claims the benefit of the priority pursuant to 35 U.S.C. §119(e) of U.S. Provisional Patent Application No. 61/185,828, filed Jun. 10, 2009, the disclosure of which is hereby incorporated by reference in its entirety.

### BACKGROUND OF THE INVENTION

The present invention relates to dual barrel syringe systems. In particular, the present invention is related to a syringe system for mixing one or more materials e.g., fluids immediately prior to or concurrently with dispensing. The present invention also relates to a syringe system that can be used to dispense a single substance or make multiple injections, dispensing multiple substances sequentially or concurrently, and for mixing multiple substances at the time of use.

Syringes or syringe-like dispensers are commonly used for dispensing various fluids/materials and for injecting such fluids. Moreover, most syringes are designed for dispensing or injecting a single fluid. However, there is an ever increasing need to dispense two different fluids that need to be isolated from each other during storage but mixed immediately prior to or concurrently with their use. In addition, there is also a need for a syringe system capable of dispensing multiple fluids, yet remains functional to be able to dispense a single fluid or multiple fluids consecutively for mixing the fluids. For example, the administration of hyaluronic acid and/or chondroiton sulfate with hyaluronidase requires that each component be stored separately and mixed immediately prior to use, delivered sequentially or in a delayed sequential manner. Such administration of compounds is required in numerous other applications, such as with certain dentifrices and epoxies.

Accordingly, there is still a need for a simple and versatile syringe-like dispenser that is capable of individually storing materials and mixing such materials immediately prior to use or delivering such materials in an immediate sequential manner.

### BRIEF SUMMARY OF THE INVENTION

In accordance with a preferred embodiment, the present invention provides a syringe assembly that includes an elongated hollow body and a first and a second cartridge. The elongated hollow body has a hub at a distal end of the hollow body and an opening at a proximal end of the hollow body. The hub includes a mixing chamber, an ejection port in fluid communication with the mixing chamber, and a first and a second cannula in fluid communication with the mixing chamber. The first and the second cartridge each independently includes a cartridge barrel body having a plunger slidably engaged with the cartridge barrel body and a seal proximate a distal end of the cartridge barrel body. The plunger also includes a plunger shaft disposed proximate the plunger. The first and the second cartridges are disposed within the elongated hollow body such that the seal is adjacent the first and the second cannula.

In accordance with another preferred embodiment, the present invention provides a dual syringe system that includes an elongated hollow body, a first and a second syringe and an end cap. The elongated hollow body has a hub at a distal end of the hollow body and an opening at a proximal end of the hollow body. The hub includes a mixing chamber, an ejection port in fluid communication with the mixing chamber, and a first and a second receiving channel in fluid communication with the mixing chamber. The first and second syringe each independently includes a syringe barrel body, a syringe tip at a distal end of the syringe barrel body, and a plunger slidably engaged with the syringe barrel body. The plunger has a plunger shaft disposed proximate the plunger. The first and the second syringes are received within the elongated hollow body and each syringe tip of the first and the second syringes is received within one of the first and second receiving channels. The end cap is connectable to the proximal end of the plunger shaft of each of the first and the second syringes.

In accordance with yet another preferred embodiment, the present invention provides a syringe system that includes an elongated hollow body, a syringe, a cartridge and an end cap. The elongated hollow body has a hub at a distal end of the hollow body. The hub includes a mixing chamber, an ejection port in fluid communication with the mixing chamber, a cannula in fluid communication with the mixing chamber, a receiving channel in fluid communication with the mixing chamber, and an opening at a proximal end of the hollow body. The syringe includes a syringe barrel body, a syringe tip at a distal end of the syringe barrel body, and a syringe plunger slidably engaged with the syringe barrel body. The syringe plunger has a syringe plunger shaft disposed proximate the syringe plunger. The syringe is disposed within the elongated hollow body such that the syringe tip is engaged with the receiving channel. The cartridge includes a cartridge barrel body, a cartridge plunger slidably engaged with the cartridge barrel body, a cartridge plunger shaft extending proximally from the cartridge plunger, and a seal at a distal end of the cartridge barrel body. The cartridge is disposed within the elongated hollow body such that the seal is adjacent the cannula. The end cap is connectable to the proximal end of the syringe plunger shaft and the cartridge plunger shaft.

In accordance with a further preferred embodiment, the present invention provides a dual cartridge dispenser that includes an elongated hollow body, a cartridge container, a first and a second cartridge and an end cap. The elongated hollow body includes a hub assembly at a distal end of the hollow body and an opening at a proximal end of the hollow body. The hub assembly has an ejection port and an inlet in fluid communication with the ejection port. The cartridge container includes a mixing hub at a distal end of the cartridge container and an opening at a proximal end of the cartridge container. The mixing hub has a mixing chamber, an outlet in fluid communication with the mixing chamber, and a first and a second cannula in fluid communication with the mixing chamber. The cartridge container is disposed within the elongated hollow body such that the outlet of the cartridge container is adjacent the inlet of the elongated hollow body. The first and the second cartridges each independently includes a cartridge barrel body having a plunger slidably engaged with the cartridge barrel body and a seal at a distal end of the cartridge barrel body. The plunger has a plunger shaft disposed proximate the plunger. The first and the second cartridges are each received within the cartridge container such that each seal of the first and the second cartridges are adjacent the first and the second cannula. The end cap is connectable to the proximal end of the plunger shaft of each of the first and the second cartridges.

In accordance with yet another preferred embodiment, the present invention provides a method for dispensing and mixing comprising the steps of providing a dispenser, providing a first material within a first cartridge, and providing a second material within a second cartridge. The dispenser includes an elongated hollow body, a first and a second cartridge, and an end cap. The elongated hollow body includes a hub at a distal end of the hollow body and an opening at a proximal end of the hollow body. The hub has a mixing chamber, an ejection port in fluid communication with the mixing chamber, and a first and a second cannula in fluid communication with the mixing chamber. The first and the second cartridges each independently includes a cartridge barrel body having a plunger slidably engaged with the cartridge barrel body and a seal at a distal end of the cartridge barrel body. The plunger has a plunger shaft disposed proximate the plunger. The first and the second cartridges are each received within the elongated hollow body such that the seals of the first and second cartridges are adjacent the first and the second cannulas. The end cap is connectable to the proximal end of the plunger shaft of each of the first and the second cartridges. The method further includes the step of dispensing the first and the second materials within the dispenser

### BRIEF DESCRIPTION OF THE SEVERAL VIEWS OF THE DRAWINGS

The foregoing summary, as well as the following detailed description of the preferred embodiments of the invention, will be better understood when read in conjunction with the appended drawings. For the purpose of illustrating the invention, there are shown in the drawings embodiments of the invention which are preferred. It should be understood, however, that the invention is not limited to the precise arrangements and instrumentalities shown.

In the drawings:

FIG. 1 is a partial, cross-sectional, perspective view, partially broken away, of a dual cartridge syringe assembly in accordance with a preferred embodiment of the present invention;

FIG. 2 is an exploded perspective view of the dual cartridge syringe assembly of FIG. 1;

FIG. 2A is a partial perspective view of a small diameter cartridge and a partial, cross-sectional, perspective view of a sleeve applicable to the dual cartridge syringe assembly of FIG. 1;

FIG. 3 is an enlarged, partial, elevational, cross-sectional view of a hub of an elongated hollow body of the dual cartridge syringe assembly of FIG. 1;

FIG. 3A is an enlarged, partial, elevational, cross-sectional view of a distal end of an alternative embodiment of an elongated hollow body applicable to the dual cartridge syringe assembly of FIG. 1;

FIG. 4 is a partial, cross-sectional, elevational view of a dummy cartridge in accordance with a preferred embodiment of the present invention;

FIG. 4A is an enlarged partial, cross-sectional, perspective view of an alternative embodiment of a dummy cartridge having grooves along a wall of the dummy cartridge;

FIG. 5 is an exploded perspective view of a dual syringe system in accordance with another preferred embodiment of the present invention;

FIG. 5A is an enlarged bottom plan view of a syringe of the dual syringe system of FIG. 5;

FIG. 6 is an enlarged partial, elevational, cross-sectional view of a hub of the elongated hollow body of the dual syringe system of FIG. 5;

FIG. 7 is a perspective view, partially broken away, of a hybrid syringe system in accordance with yet another preferred embodiment of the present invention;

FIG. 8 is an exploded perspective view of the hybrid syringe system of FIG. 7;

FIG. 9 is an enlarged partial, elevational, cross-sectional view of a hub of the hybrid syringe system of FIG. 7;

FIG. 10 is an enlarged, partial, elevational, cross-sectional view of a distal end of a dual cartridge dispenser in accordance with yet another preferred embodiment of the present invention;

FIG. 11 is a flow diagram of a method for concurrently dispensing and mixing two fluids in accordance with yet another preferred embodiment of the present invention;

FIG. 12 is a partially elevational view of a cartridge and a cross-sectional elevational view of a needle hub assembly assembled to the cartridge in accordance with another preferred embodiment of a dual cartridge syringe assembly of the present invention; and

FIG. 13 is an enlarged, partial, cross-sectional, elevational view of a distal end of an elongated hollow body of a dual cartridge syringe assembly for use with the needle hub assembly of FIG. 12.

### DETAILED DESCRIPTION OF THE INVENTION

Certain terminology is used in the following description for convenience only and is not limiting. The words “right,” “left,” “lower” and “upper” designate directions in the drawings to which reference is made. The words “inwardly” and “outwardly” refer to directions toward and away from, respectively, the geometric center of the identified element and designated parts thereof. Additionally, the term “a,” as used in the specification, means “at least one.” The terminology includes the words noted above, derivatives thereof and words of similar import.

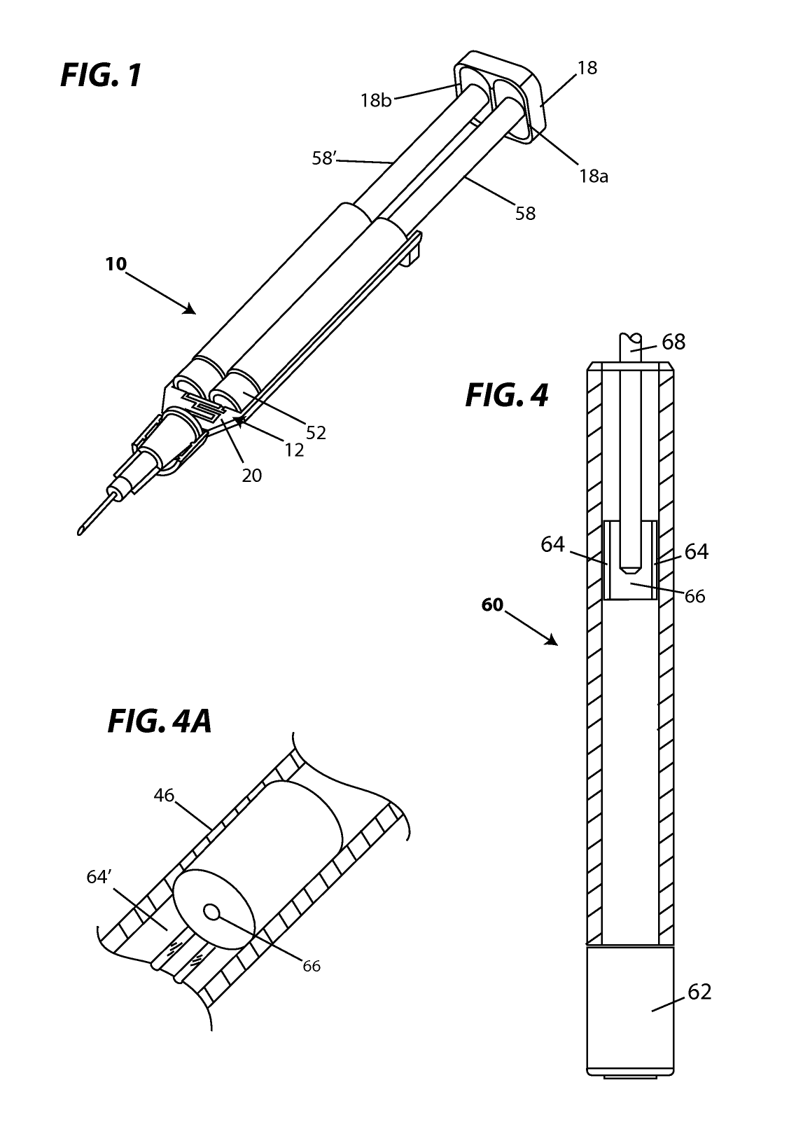

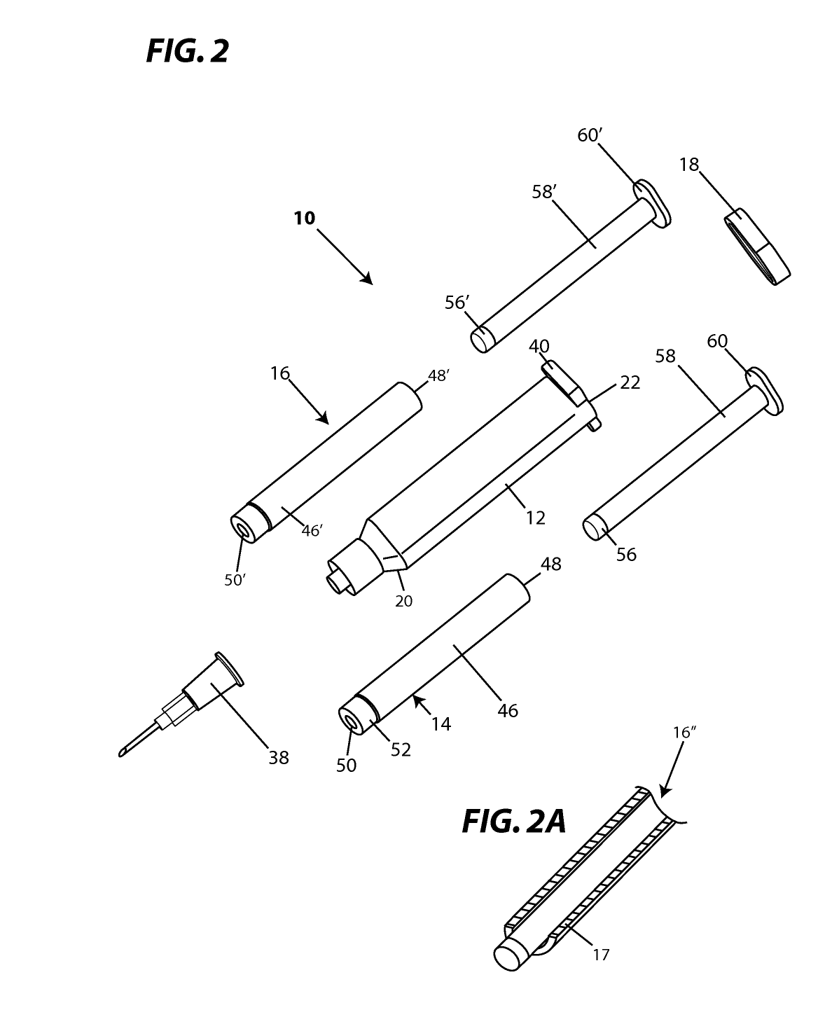

FIG. 1 illustrates a preferred dual cartridge syringe assembly 10 embodiment of the present invention in an assembled configuration. As best shown in FIG. 2, the dual cartridge syringe assembly 10 includes an elongated hollow body 12, a first and a second cartridge 14, 16 and an end cap 18.

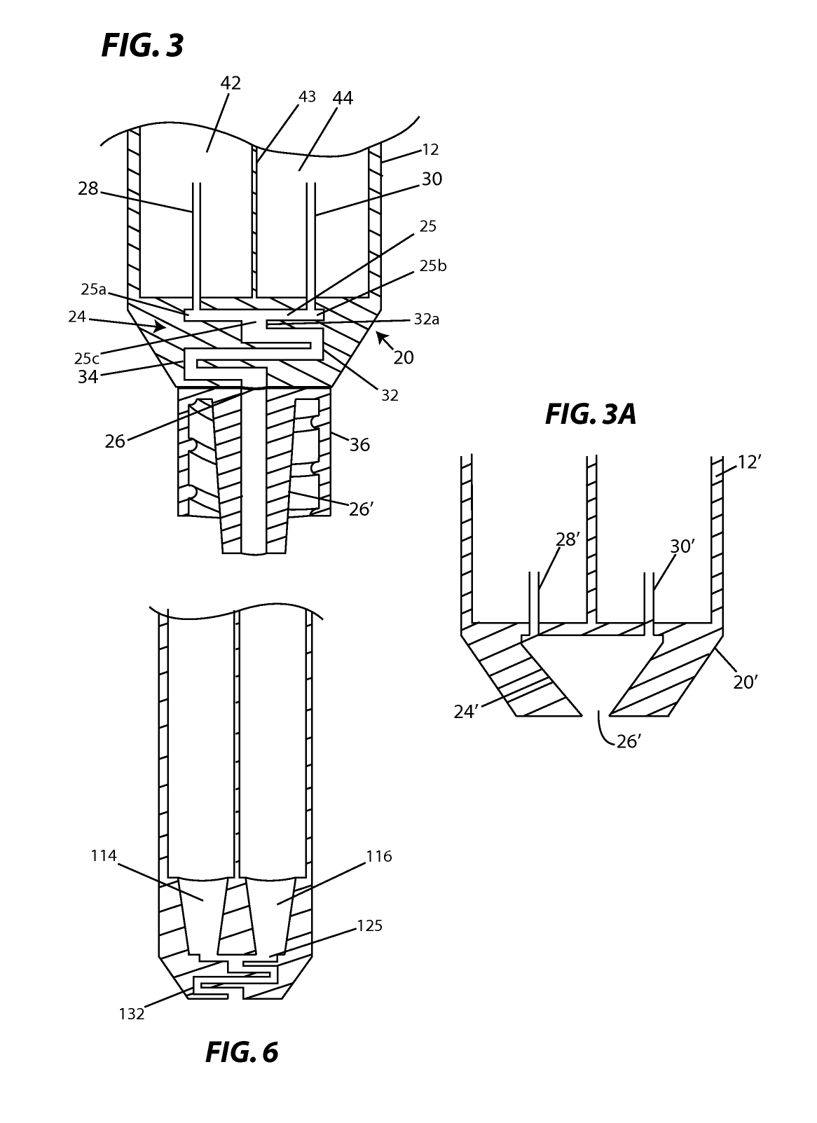

The elongated hollow body 12 includes a hub 20 at a distal end of the elongated hollow body 12 and an opening 22 at a proximal end of the elongated hollow body 12. As shown in FIG. 3, the hub 20 is preferably formed integrally as part of the elongated hollow body 12. However, the hub 20 can alternatively be configured as a separate component releasably or permanently connected to the elongated hollow body 12.

The elongated hollow body 12 can be formed as an integrally molded component. However, the elongated hollow body 12 can alternatively be formed as an two separate halves, for example, a front half (as shown in FIGS. 3, 6 and 9) and a back half (e.g., minor opposites of the halves shown in FIGS. 3, 6 and 9) that are assembled together to form the elongated hollow body 12. The assembly of the front and back halves can be accomplished by a snap fit connection, an adhesive, or any other means readily known in the art. Formation of the elongated hollow body 12 by respective front and back halves advantageously facilitates the manufacture and assembly of the elongated hollow body 12.

The hub 20 includes a mixing chamber 24 and an ejection port 26. The mixing chamber 24 can be a chamber of any configuration sufficient to receive two fluids wherein mixing can occur, such as a cube shaped chamber, a spherical chamber, or the like. Preferably, the mixing chamber 24 is configured as an elongated plenum 25 in communication with the ejection port 26. More preferably, the mixing chamber 24 includes a winding elongated channel chamber 32 having one or more bends 34. The bends 34 advantageously induce mixing of materials entering the mixing chamber 24 with each other prior to being expelled from the mixing chamber 24 through the ejection port 26. Preferably, the mixing chamber 24 includes at least two bends 34, more preferably bends 34 that are configured as substantially ninety degree bends.

The plenum 25 includes a first end 25a, a second end 25b and an outlet 25c. The outlet 25c is positioned to be substantially equidistant from the first end 25a and the second end 25b. Positioning the outlet 25c to be equidistant from the first and second ends 25a, 25b advantageously allows for material flow entering from both the first and second ends 25a, 25b to substantially meet within a center region of the plenum 25 and thus concurrently flow out through the outlet 25c. When the mixing chamber 24 includes the winding elongated channel chamber 32, the outlet 25c is in communication with an inlet 32a of the elongated winding channel 32.

The ejection port 26, as shown in FIG. 3, is an opening in fluid communication with the mixing chamber 24 proximate the distal end of the hub 20. The ejection port 26 can alternatively be configured to receive and removably connect to a luer adapter 36, a luer lock, or the like for attachment to an external cannula, such as a needle 38 (e.g., a hypodermic needle as shown in FIG. 2) or a cannula, including a metal cannula or a blunt plastic cannula. The connection of the luer adapter 36 to the hub 20 can be by any well known manner known in the art, thus a detailed description of such connections is not necessary for a complete understanding of the present invention Alternatively, the ejection port 26 can be configured for direct attachment to an external cannula. For example, the ejection port 26 can be configured with female threads for mating with male threads or as a taper lock for receiving a corresponding male counterpart. The ejection port 26 can also be configured with a nozzle 26′ that extends outwardly from the hub 20, as shown in FIG. 3. The ejection port 26 is not limited to such configurations for connecting or attaching to a luer adapter or the like, but can be of any configuration sufficient to form a rigid attachment with such adapters.

The dual cartridge syringe assembly 10 also includes a first and a second cannula 28, 30 that each extends proximally from the hub 20 within the elongated hollow body 12. The first and second cannulas 28, 30 are each independently in fluid communication with the mixing chamber 24 and extend proximally away from the mixing chamber 24. The first and second cannulas 28, 30 can be any cannulas readily known in the art or to be developed which are capable of piercing a resealable seal. For example, the cannulas 28, 30 can each be a metal cannula or a plastic cannula. Preferably, the first and second cannulas 28, 30 are positioned within the hollow body 12 such that the first cannula 28 is in communication with the first end 25a and the second cannula 30 is in communication with second end 25b of the plenum 25. The first and second cannulas 28, 30 can be attached to the mixing chamber 24, for example by an adhesive, a detent, or formed integrally with the hub 20.

The elongated hollow body 12 can optionally include a flange 40 at the proximal end, as shown in FIG. 2. Furthermore, the elongated hollow body 12 can optionally be configured with an internal geometry shaped as two elongated cavities or chambers i.e., a first chamber 42 and a second chamber 44 defining a dual chamber body, as shown in FIG. 3, for receiving a separate cartridge within each individual chamber. The dual chamber body can be formed, for example, by a wall 43 that extends in the axial direction within the elongated hollow body 12. When the hollow body 12 is configured with the first and second chambers 42, 44, the first chamber 42 is in communication with the first cannula 28 while the second chamber 44 is in communication with the second cannula 30.

FIG. 3A illustrates an alternative embodiment of the present invention with an elongated hollow body 12′ having a hub 20′. The hub 20′ includes first and second cannulas 28′, 30′, similar to cannulas 28, 30, in communication with a mixing chamber 24′. The mixing chamber 24′ is configured to have a cross-sectional profile substantially of an inverted triangle. In other words, the mixing chamber 24′ can be configured as an inverted cone, an inverted pyramid, an inverted frustrum of a cone, or the like. The hub 20′ further includes an ejection port 26′ in communication with the mixing chamber 24′ but does not include a winding elongated plenum 25 as described above.

The first and second cartridges 14, 16 are identical and the following detailed description of one cartridge (e.g., the first cartridge 14) applies to the second cartridge 16. For convenience, second cartridge reference numerals are the same as the first cartridge reference numerals but with the addition of prime “′”. As shown in FIG. 2, the first cartridge 14 includes a hollow cartridge barrel body 46 of a generally tubular configuration having a proximal end and a distal end. The proximal end of the cartridge barrel body 46 has an opening 48. The distal end of the cartridge barrel body 46 has a pierceable and resealable seal 50 connectable to the cartridge barrel body 46 by, for example, a cap 52. The cap 52 and seal 50 can be of any known seal and cap configuration and a detailed description of these elements is not necessary for a complete understanding of the present embodiment. However, exemplary seals 50 can be made from silicone, elastomers, such as rubber or thermoplastic elastomers, or any other material know in the art or to be developed which would be suitable for its intended purpose.

The first cartridge 14 is configured to receive a plunger 56 through the opening 48. The plunger 56 slidingly engages with an interior surface of the cartridge barrel body 46 in a manner well known in the art. The plunger 56 has a plunger shaft 58 that extends proximally from the plunger 56. The plunger shaft 58 can optionally include a flange 60 at the most proximal end of the plunger shaft 58.

The plunger shaft 58 and/or the flange 60 can be color coded to facilitate identification of the contents of the cartridge or which cartridge should be injected first, if used in a sequential manner. For example, the plunger shaft 58 can be colored green, while the plunger shaft 58′ can be colored red. Alternatively, any other color in addition to green and red can be used to color code the plunger shafts 58, 58′ and/or flanges 60, 60′.

The present embodiment of the dual cartridge syringe assembly 10 can optionally include an end cap 18. The end cap 18 is preferably configured, as best shown in FIG. 1 to receive the proximal ends (flanges 60, 60′ if provided) of the plunger shafts 58, 58′ of the first and second cartridges 14, 16. The end cap 18 is connectable to the plunger shafts 58, 58′ and can be readily removed from or rotated about either plunger shaft 58 or plunger shaft 58′. The end cap 18 is configured with at least one recess 18a and preferably two recess 18a, 18b and a snap-fit, taper-fit, dove-tail, or the like for connecting to the proximal ends of the plunger shafts 58, 58′. Although end cap 18 is preferred for use with the present embodiment, any other means sufficient to allow both plunger shafts 58, 58′ to be depressed substantially simultaneously such that respective plunger shafts 58, 58′ concurrently dispense material from the cartridges 46, 46′ can be used. For example, a rigid attachment of the distal ends or mid portions of the plunger shafts 58, 58′ themselves, without the use of an additional cap like component, can serve as a means for allowing both plunger shafts 58, 58′ to be cooperatively or substantially simultaneously depressed.

The end cap 18 can alternatively be a removably and selectively engageable end cap 18 for connecting to at least one of the plunger shafts 58, 58′. That is, the selectively engageable end cap 18 can be selected to engage only a single plunger shaft e.g., plunger shaft 58, and then subsequently selected to engage another plunger shaft e.g., plunger shaft 58′. For example, when assembled to plunger shaft 58, the end cap 18 is rotatable thereon to move between engagement with a single plunger shaft (e.g., 58) and two plunger shafts (e.g., 58, 58′). Alternatively, the end cap 18 can be configured to slide from one plunger shaft to another or from engagement of a single plunger shaft to engagement of two plunger shafts. Thus, the present embodiment advantageously provides for a dual cartridge syringe assembly 10 that can operate to dispense injectables, such as hyaluronic acid and/or chondroiton sulfate and hyaluronidase from within the cartridges 46, 46′, respectively, either simultaneously, as a single injectable from a single cartridge, or for dispensing multiple injections sequentially.

In an assembled state, the first and second cartridges 14, 16 each have their distal ends sealed with seals 50, 50′, which form a fluid tight seal. The plungers 56, 56′ are slidably inserted into the openings 48, 48′ of the cartridge barrel bodies 46, 46′ for both the first and second cartridges 14, 16. The first and second cartridges 14, 16 are then inserted into the elongated hollow body 12 through the opening 22 such that the plunger shafts 58, 58′ of each of the cartridges 46, 46′ extend from the elongated hollow body 12 (see FIG. 1). Moreover, the first and second cartridges 14, 16 are initially positioned within the elongated hollow body 12 with the seals 50, 50′ either in contact with or spaced apart from the cannulas 28, 30. In other words, in the initial assembled state, the seals 50, 50′ are not pierced by the cannulas 28, 30. The end cap 18 is then connected to proximal ends of the plunger shafts 58, 58′. The present embodiment therefore advantageously provides for a modular syringe assembly wherein a user can easily insert and remove various cartridges and/or utilize a needle or other cannula attachment for effectuating a mixing of one or more polymers or other substances and the dispensing of the mixed substances.

In operation, the dual cartridge syringe assembly 10 is assembled with a first fluid disposed within the first cartridge 14 and a second fluid disposed within the second cartridge 16. The use of the term “fluid” is given its broadest possible interpretation and includes all fluids such as liquids, gases, and any other materials with fluid properties. A user then depresses the end cap 18 which drives the plunger shafts 58, 58′ distally relative to the cartridges 46, 46′. The distal movement of the plunger shafts 58, 58′ also drive the first and second cartridges 14, 16 distally such that the seals 50 50′ are pierced by the proximal ends of the first and second cannulas 28, 30. Upon further depression of the end cap 18 by the user, the first and second fluids are concurrently driven out of the first and second cartridges 14, 16, through the first and second cannulas 28, 30, and into the mixing chamber 24 where they are mixed, and then passed out through the ejection port 26. Thus, a mixing of the first and second fluids immediately prior to or concurrently with dispensing is accomplished. Accordingly, as a result of the present embodiment, the dual cartridge syringe assembly 10 provides co-injection/mixing capabilities that result in the dispensing of a freshly mixed mixture.

The dual cartridge syringe assembly 10 can be used to dispense equal or unequal volumes from the respective first and second cartridges 14, 16, such that a larger first volume of a first liquid in the first cartridge 14 is uniformly mixed with a smaller second volume of a second liquid in the second cartridge 16. This can be accomplished with a thinner i.e., smaller inside diameter second cartridge 16″ (FIG. 2A) which may also be configured with the same or smaller outside diameter than the first cartridge 14 and an overall axial length that is the substantially same or smaller than that of the first cartridge 14. That is, the small diameter second cartridge 16″, having a smaller lumen and substantially the same (or smaller) length compared to the first cartridge, can be configured as an overall thinner cartridge or a thick-walled cartridge having an outside diameter the same diameter as the first cartridge, but with a smaller inside diameter. As such, the dual cartridge syringe assembly 10 can facilitate the proper mixing of different components when both plungers are pushed simultaneously.

The small diameter second cartridge 16″ can optionally be used in conjunction with a sleeve 17 (FIG. 2A) to facilitate a secure assembly of the elongated hollow body 12 and minimize play within the elongated hollow body 12 during use.

In addition, the user has the option of using the dual cartridge syringe assembly 10 to dispense only a single fluid. This can be accomplished by inserting two cartridges having the same contents, by inserting only a single cartridge (e.g., cartridge 14) within the elongated hollow body 12, by twisting or rotating the end cap 18 such that only a single cartridge plunger is engaged with the end cap 18, or by using a “dummy” or empty cartridge. By rotating the end cap 18, a user can select a single cartridge plunger for dispensing. In addition, by selecting only a single cartridge at a time for dispensing, the present embodiment advantageously provides for a means to essentially immediately and sequentially dispense one or more fluids.

A dummy cartridge 60 applicable to the present embodiment is shown in FIG. 4. The dummy cartridge 60 is similar to the first and second cartridges 14, 16, except for the inclusion of an air channel 64 formed within a plunger 66. Like the seal 50 of the first cartridge 14, dummy cartridge 60 includes a stopper 62 made from a pierceable and resealable material. In addition, the dummy cartridge 60 includes a plunger shaft 68 that is proximate to the plunger 66 and extends proximately from the plunger 66. The plunger 66 includes at least one air channel 64 and preferably a plurality of air channels 64 that run axially through the plunger 66 to allow air or entrapped gases to exit the cartridge 60 while the plunger 66 is being depressed. That is, the air channels 64 provide fluid communication between an area proximate a distal surface of the plunger 66 and an area proximate a proximal surface of the plunger 66.

In an alternative embodiment, instead of the plunger 66 being configured with axially extending air channels 64, the dummy cartridge 60 can be configured with air channels 64′ (FIG. 4A) that run axially along the length of the dummy cartridge 60 on the cartridge barrel body 46 to allow air or entrapped gases to exit while the plunger 66 is depressed. Thus, the present embodiment advantageously provides for a syringe system 10 that can dispense multiple fluids concurrently, a single fluid individually, or multiple fluids sequentially, as required by the user.

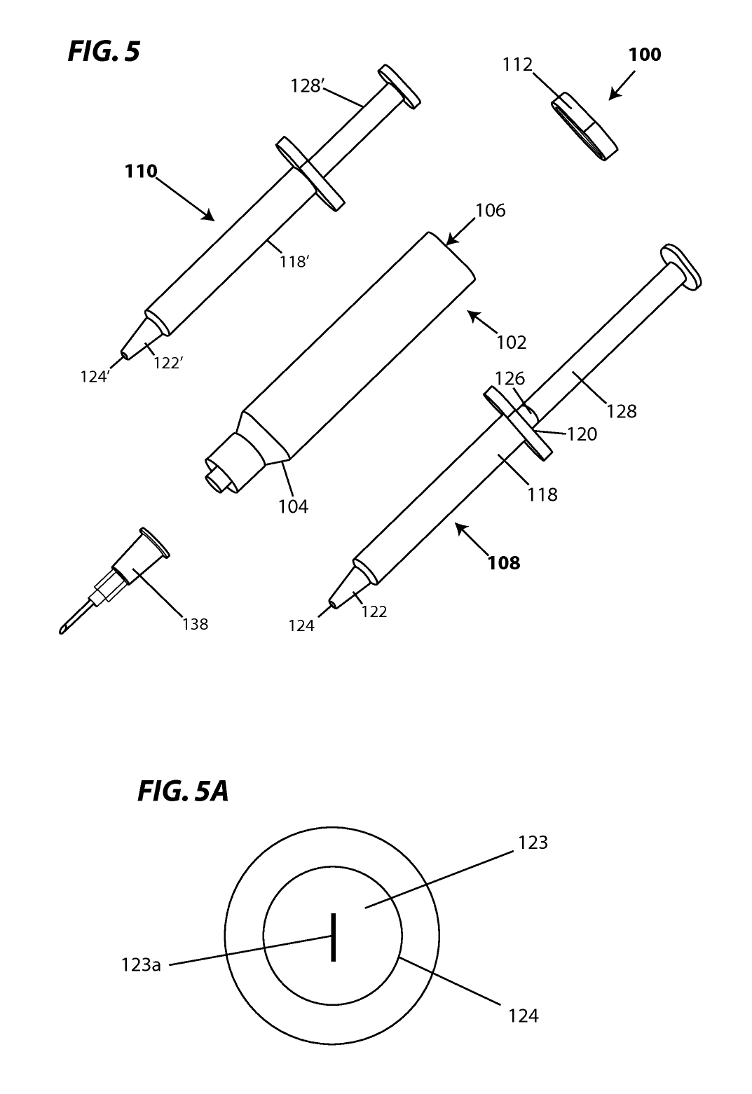

In another preferred embodiment, the present invention provides for a dual syringe system 100 having two syringe bodies 108, 110. As shown in FIG. 5, the syringe system 100 includes an elongated hollow body 102 that includes a hub 104 proximate a distal end of the hollow body 102 and an opening 106 at a proximal end of the hollow body 102. The syringe system 100 also includes first and second syringes 108, 110 and an end cap 112. The end cap 112 is as described in the prior embodiment and the elongated hollow body 102 is as described in the prior embodiment except for the hub 104. The hub 104 is similar to the hub 20 as described in the prior embodiment except that the hub 104 has first and second receiving channels 114, 116, as shown in FIG. 6, instead of first and second cannulas 28, 30.

The first and second syringes 108, 110 are identical and a detailed description of one syringe (e.g., the first syringe 108) applies to the second syringe 110. For convenience, second syringe reference numerals are the same as the first syringe reference numerals but with the addition of prime “′”. As shown in FIG. 5, the first syringe 108 includes a syringe barrel body 118 having a proximal end and a distal end. The proximal end of the syringe barrel body 118 has an opening 120. The distal end of the syringe barrel body 118 has a syringe tip 122. The syringe tip 122 is generally conical in shape, tapering toward its distal end. The most distal end of the syringe tip 122 has an aperture 124 to allow fluid to exit the syringe 108. Alternatively, the syringe tip 122 can be of any configuration, such as a frustum, frustro-conical, pyramidal, cylindrical, etc., or any other configuration sufficient for its intended use. The first syringe 108 further includes a plunger 126 and a plunger shaft 128, as described in the prior embodiment. The syringe tip 122 can optionally further include an openable seal 123 (FIG. 5A) that maintains fluid within the syringe 108, but allows fluid to be expelled upon the application of the plunger (e.g., pressure applied to the plunger 126 to expel fluid from the syringe 108). The openable seal 123 can be, for example, a silicone or elastomer seal having a slit 123a.

In an assembled state, the present embodiment is assembled as in the prior embodiment except for the first and second syringes 108, 110. In the present embodiment, the syringe tips 122, 122′ of the first and second syringes 108, 110, when assembled into the elongated hollow body 102 engage with the first and second receiving channels 114, 116 (FIG. 6). Preferably, the syringe tips 122, 122′ of the first and second syringes 108, 110 are complementary in shape to the first and second receiving channels 114, 116, such that the syringe tip apertures 124, 124′ are fixed in position with little or no play within their respective receiving channels 114, 116. As used herein, the term “play” means little or slight movement. The operation of the present embodiment's dual syringe system 100 is the same as described for the prior embodiment.

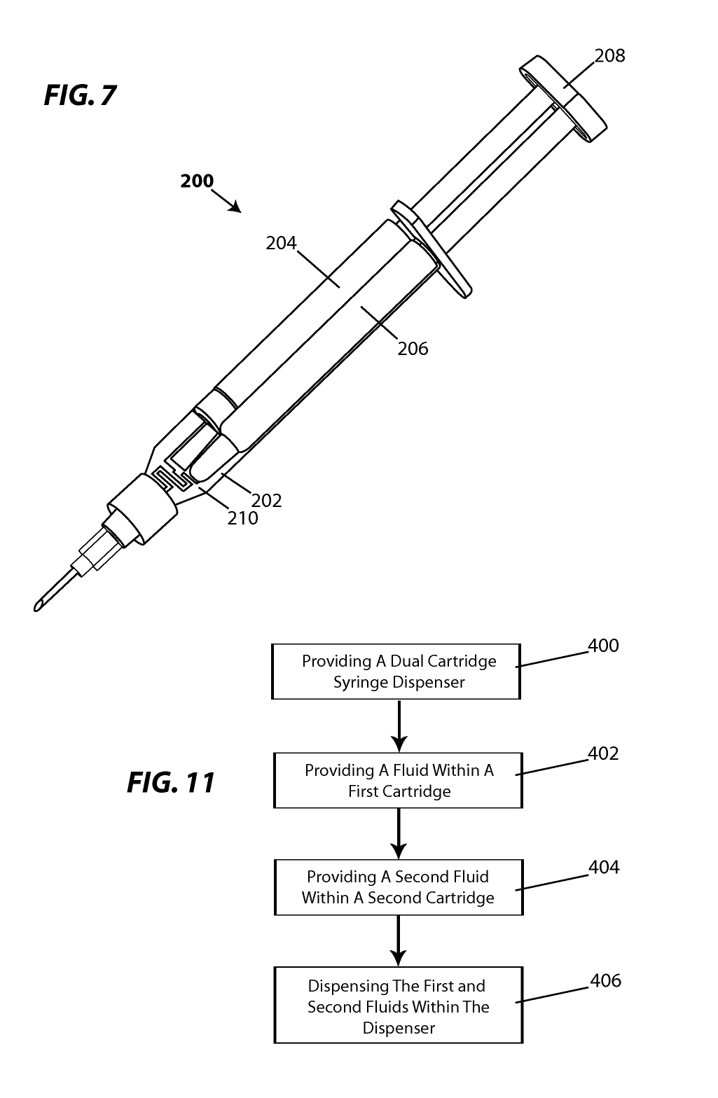

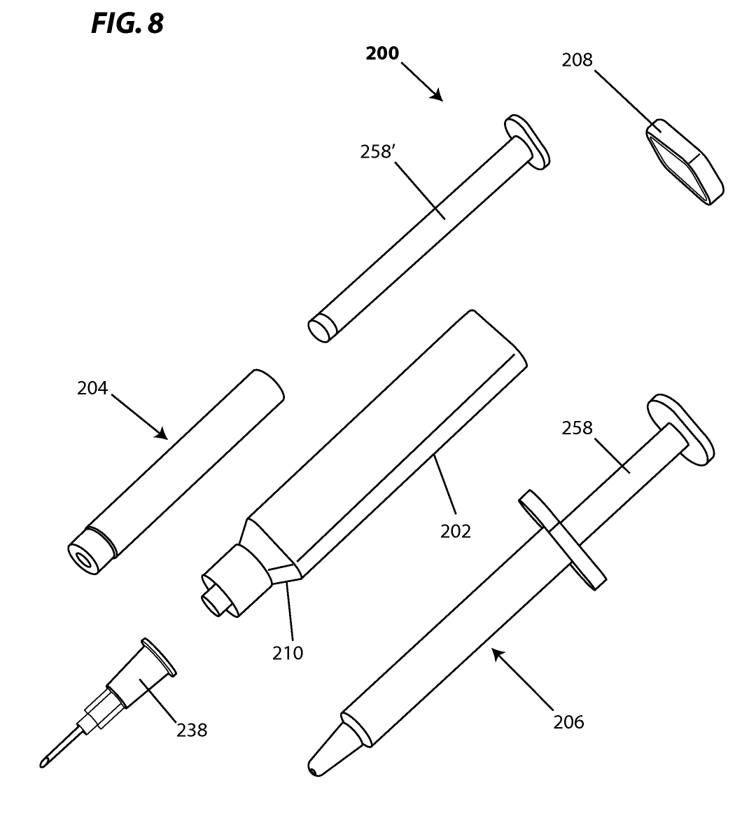

In yet another preferred embodiment, the present invention provides for a hybrid syringe system 200, as shown in FIGS. 7-9. This embodiment is a hybrid of the prior two embodiments. As shown in FIG. 8, the hybrid syringe system 200 includes an elongated hollow body 202, a cartridge 204, a syringe 206 and an end cap 208.

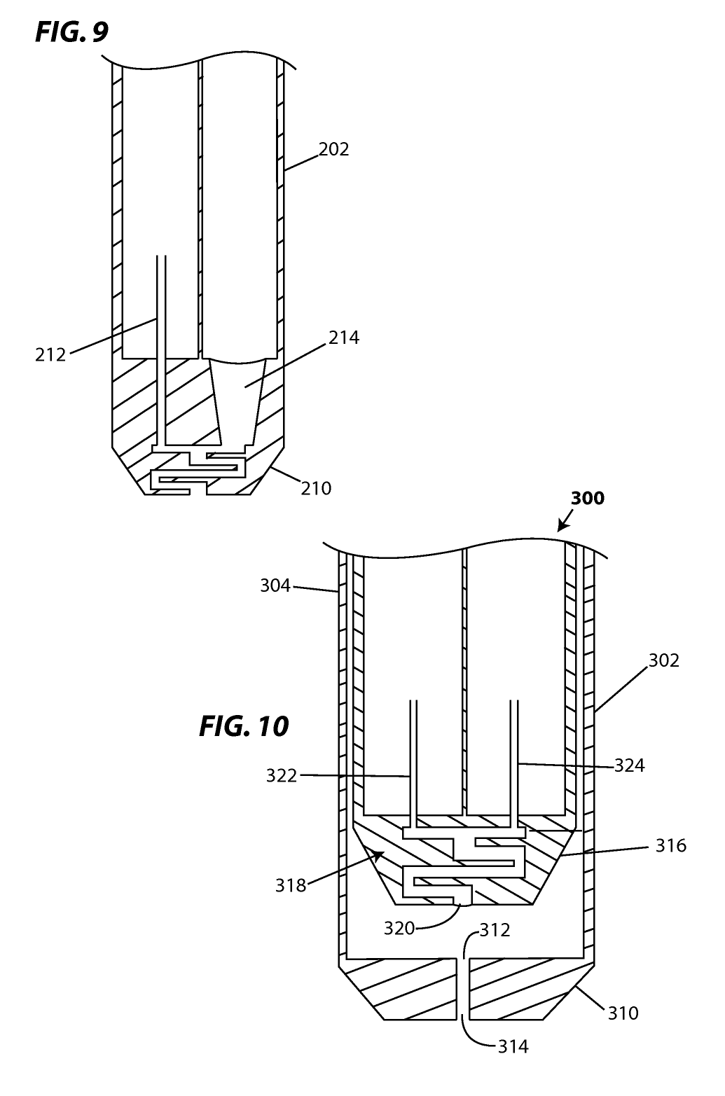

The elongated hollow body 202 includes a hub 210, as shown in FIG. 9. The hub 210 includes both a single cannula 212, as previously described in the dual cartridge syringe assembly 10 embodiment above, and a single receiving channel 214, as previously described in the dual syringe system 100 embodiment above. Accordingly, the assembly of and operation of the hybrid syringe system 200 is analogous to those of the prior embodiments described above and a further detailed description of its assembly or operation is not necessary for a complete understanding of the present embodiment.

In a further embodiment, the present invention provides for a dual cartridge dispenser 300, as shown in FIG. 10. The dual cartridge dispenser 300 includes an elongated hollow body 302, a cartridge container 304, a first and a second cartridge (not shown) and an end cap (not shown).

The elongated hollow body 302 is configured to receive the dual cartridge dispenser 300 and includes a hub 310 at a distal end of the hollow body 302 and an opening (not shown) at a proximal end of the hollow body 302. As shown in FIG. 10, the hub 310 has an inlet 312 and an ejection port 314 that is in fluid communication with the inlet 312. The ejection port 314 is similar to the ejection port 26 described in the above dual cartridge syringe assembly 10 embodiment. The inlet 312 can alternately be an inlet of a nozzle or a cannula (not shown) that extends distally from the hub 310.

The cartridge container 304 includes a mixing hub 316 having a mixing chamber 318 similar to those described in any of the above embodiments, an outlet 320 in fluid communication with the mixing chamber 318, and first and second cannulas 322, 324 that are each independently in fluid communication with the mixing chamber 318. The first and second cannulas 322, 324 are similar to the first and second cannulas as described in the above dual cartridge syringe assembly 10 embodiment. The first and second cartridges 302, 304 and end cap are as described in the dual cartridge syringe assembly 10 embodiment above.

A further aspect of the present invention is a method for dispensing and mixing at least two fluids, as shown in FIG. 11. The method includes the steps of providing a dispenser as described in any of the above described embodiments of the present invention, such as the dual cartridge syringe assembly embodiment (step 400). With respect to the dual cartridge syringe assembly, the method further includes the step of providing a first fluid within the first cartridge (step 402) and providing a second fluid within the second cartridge (step 404). Then, dispensing the first and the second fluids within the dispenser by actuating the dispenser provided (e.g., by depressing upon the end cap) (step 406). The dispensing of the first and second fluids can be accomplished substantially concurrently with, for example, the use of an end cap attached to both the first and second plunger rods of the dual syringe cartridge system. The dispensing can alternatively be accomplished sequentially by dispensing the first fluid within the first cartridge and then immediately dispensing the second fluid within the second cartridge or by dispensing the first and second fluids in steps e.g., in a delayed sequential manner. This can be accomplished by removing the end cap to the dual cartridge syringe assembly and dispending each individual cartridge in a sequential manner.

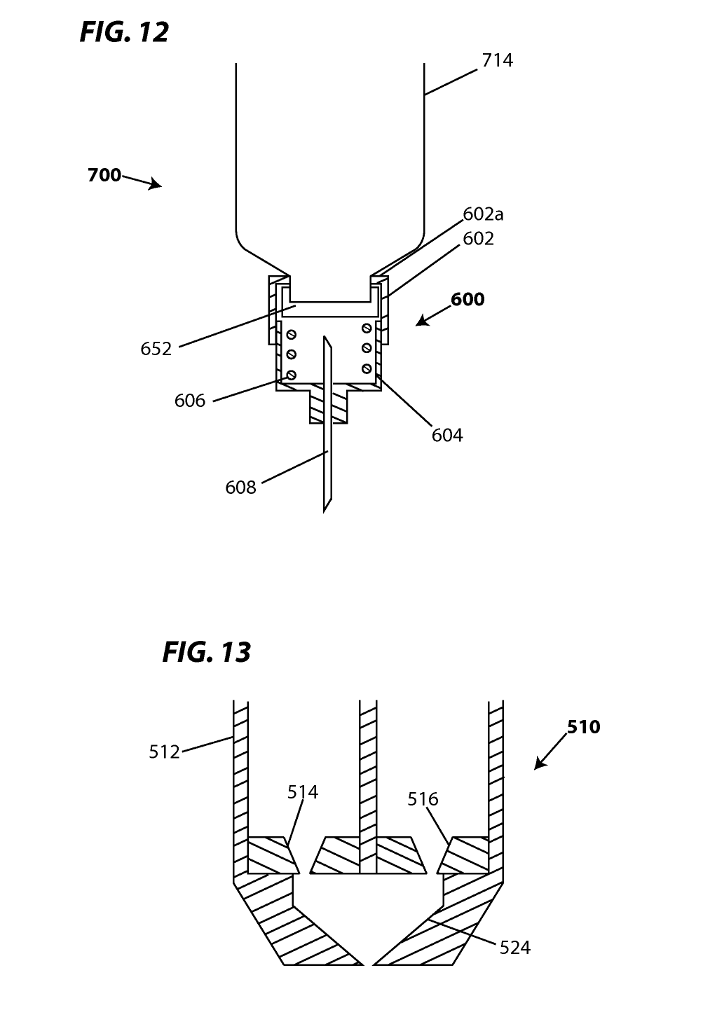

In yet another preferred embodiment, the present invention provides for a dual syringe assembly 510, as shown in FIGS. 12 and 13. The dual syringe assembly 510 has an elongated hollow body 512, similar to the elongated hollow body 102 of the dual syringe system 100, except for the configuration of the mixing chamber 524. The mixing chamber 524 is configured similarly to the mixing chamber 24′ of FIG. 3A. The elongated hollow body 512 also includes receiving channels 514, 516, similar to receiving channels 114, 116, as described above. In particular, receiving channels 514, 516 are generally each configured as a distally tapering channel to facilitate insertion of the needle 608 of the needle hub assembly 600 into the mixing chamber 524.

The dual syringe assembly 510 is preferably configured for use with a cartridge assembly 700 having a cartridge 714, similar to cartridge 14 described above, and a needle hub assembly 600. The needle hub assembly 600 is attachable to a cap portion 652 of the cartridge 714. The needle hub assembly 600 includes a substantially tubular main body portion 602, a needle hub assembly 604 slidably attached to the main body portion 602, and a biasing member 606 disposed between the main body portion 602 and the needle hub assembly 604. The main body portion 602 is attachable to the cap portion 652 by a radially inward flange 602a that engages an outer edge of the cap portion 652. The biasing member 606 maintains the main body portion 602 in a spaced apart relation with respect to the needle hub assembly 604. The main body portion 602 and the needle hub assembly 604 are retained together against the force of the biasing member 606 by cooperating catches, such as flanges or detents. The biasing member 606 can be any conventional biasing member such as a compression spring.

In operation, one or two cartridge assemblies 700 is inserted within the dual syringe assembly 510. In an assembled and pre-injection state, the needle 608 is received within e.g., the receiving channel 514 and guided to the mixing chamber 524 owing to the distally tapering configuration of the receiving channel 514. Then, when an injection is to be made, a user depresses upon a plunger rod (not shown) attached to the cartridge assembly 700 in a manner similar to that described in any of the above embodiments. Depression of the plunger rod drives the cartridge 714 distally such that the needle hub assembly 604 bottoms out against as surface of the receiving channel 514, thereby allowing for the compression of the needle hub assembly 600. Compression of the needle hub assembly 600 forces the proximal end of the needle 608 to pierce the cartridge's seal (not shown) within the cap portion 652 and allow fluid communication for the flow of the cartridge's contents through the needle 608 and into the mixing chamber 524.

It will be appreciated by those skilled in the art that changes could be made to the embodiments described above without departing from the broad inventive concept thereof. It is understood therefore, that this invention is not limited to the particular embodiments disclosed, nor limited to medical or industrial uses, but it is intended to cover modifications within the spirit and scope of the present invention as described above.

I claim:

1. A syringe assembly comprising:
an elongated hollow body that includes:
a hub at a distal end of the hollow body, the hub having:
a mixing chamber,
an ejection port in fluid communication with the mixing chamber, and
a first and a second cannula in fluid communication with the mixing chamber, and
an opening at a proximal end of the hollow body; and
a first and a second cartridge each independently including:
a cartridge barrel body having:
a plunger slidably engaged with the cartridge barrel body, the plunger having a plunger shaft disposed proximate the plunger, and
a seal proximate a distal end of the cartridge barrel body,
wherein the first and the second cartridges are disposed within the elongated hollow body such that the seal is adjacent the first and the second cannula.

2. The syringe assembly of claim 1, further comprising an end cap connectable to the proximal end of at least one of the first and the second plunger shafts.

3. The syringe assembly of claim 1, further comprising an end cap selectively engageable to connect to at least one of the first and the second plunger shafts.

4. The syringe assembly of claim 1, further comprising a luer adapter or a cannula connectable to the ejection port.

5. The syringe assembly of claim 1, wherein the elongated hollow body is a dual chamber body having a wall extending in an axial direction within the elongated hollow body forming a first chamber in communication with the first cannula and a second chamber in communication with the second cannula.

6. The syringe assembly of claim 1, wherein the mixing chamber comprises an elongated winding channel.

7. The syringe assembly of claim 6, wherein the elongated winding channel includes at least one bend about ninety degrees.

8. The syringe assembly of claim 1, wherein the mixing chamber comprises:
an elongated plenum that includes:
a first end in communication with the first cannula,
a second end in communication with the second cannula, and
an outlet substantially equidistant from the first end and the second end; and
an elongated winding channel in communication with the outlet and the ejection port.

9. The syringe assembly of claim 1, wherein the seal of one of the first and second cartridges includes an air channel extending axially through the plunger to allow fluid communication from an area proximate a distal surface of the plunger to an area proximate a proximal surface of the plunger.

10. The syringe assembly of claim 1, wherein the first cartridge has a first volume and a first length and the second cartridge has a second volume smaller than the first volume and a second length substantially the same as the first length.

11. A dual syringe system comprising:
an elongated hollow body that includes:
a hub at a distal end of the hollow body, the hub having:
a mixing chamber,
an ejection port in fluid communication with the mixing chamber, and
a first and a second receiving channel in fluid communication with the mixing chamber, and
an opening at a proximal end of the elongated hollow body;
a first and a second syringe each independently including:
a syringe barrel body,
a syringe tip at a distal end of the syringe barrel body, and
a plunger slidably engaged with the syringe barrel body, the plunger having a plunger shaft disposed proximate the plunger,
wherein the first and the second syringes are received within the elongated hollow body and each syringe tip of the first and the second syringes is received within one of the first and second receiving channels; and
an end cap connectable to the proximal end of the plunger shaft of each of the first and the second syringes.

12. The dual syringe system of claim 11, wherein the mixing chamber comprises an elongated winding channel.

13. The dual syringe system of claim 12, wherein the elongated winding channel includes at least one bend about ninety degrees.

14. The dual syringe system of claim 11, wherein the mixing chamber comprises:
an elongated plenum that includes:
a first end in communication with the first receiving channel,
a second end in communication with the second receiving channel, and
an outlet substantially equidistant from the first end and the second end; and
an elongated winding channel in communication with the outlet and the ejection port.

15. A syringe system comprising:
an elongated hollow body that includes:
a hub at a distal end of the hollow body, the hub having:
a mixing chamber,
an ejection port in fluid communication with the mixing chamber,
a cannula in fluid communication with the mixing chamber, and
a receiving channel in fluid communication with the mixing chamber, and
an opening at a proximal end of the hollow body;
a syringe that includes:
a syringe barrel body,
a syringe tip at a distal end of the syringe barrel body, and
a syringe plunger slidably engaged with the syringe barrel body, the syringe plunger having a syringe plunger shaft disposed proximate the syringe plunger,
wherein the syringe is disposed within the elongated hollow body such that the syringe tip is engaged with the receiving channel;
a cartridge that includes:
a cartridge barrel body,
a cartridge plunger slidably engaged with the cartridge barrel body,
a cartridge plunger shaft extending proximally from the cartridge plunger, and
a seal at a distal end of the cartridge barrel body,
wherein the cartridge is disposed within the elongated hollow body such that the seal is adjacent the cannula; and
an end cap connectable to the proximal end of the syringe plunger shaft and the cartridge plunger shaft.

16. The syringe system of claim 15, wherein the mixing chamber comprises an elongated winding channel.

17. The syringe system of claim 15, wherein the elongated winding channel includes at least one bend about ninety degrees.

18. The syringe system of claim 15, wherein the mixing chamber comprises:
an elongated plenum that includes:
a first end in communication with the cannula,
a second end in communication with the receiving channel, and
an outlet substantially equidistant from the first end and the second end; and
an elongated winding channel in communication with the outlet and the ejection port.

19. A dual cartridge dispenser comprising:
an elongated hollow body that includes:
a hub assembly at a distal end of the hollow body, the hub having:
an ejection port, and
an inlet in fluid communication with the ejection port, and
an opening at a proximal end of the hollow body; and
a cartridge container that includes:
a mixing hub at a distal end of the cartridge container, the mixing hub having:
a mixing chamber,
an outlet in fluid communication with the mixing chamber, and
a first and a second cannula in fluid communication with the mixing chamber, and
an opening at a proximal end of the cartridge container,
wherein the cartridge container is disposed within the elongated hollow body such that the outlet of the cartridge container is adjacent the inlet of the elongated hollow body;
a first and a second cartridge each independently including:
a cartridge barrel body having:
a plunger slidably engaged with the cartridge barrel body, the plunger having a plunger shaft proximate the plunger, and
a seal at a distal end of the cartridge barrel body,
wherein the first and the second cartridges are each received within the cartridge container such that each seal of the first and the second cartridges are adjacent the first and the second cannula; and
an end cap connectable to the proximal end of the plunger shaft of each of the first and the second cartridges.

20. A method for dispensing and mixing comprising the steps of:
providing a dispenser that includes:
an elongated hollow body that includes:
a hub at a distal end of the hollow body, the hub having:
a mixing chamber,
an ejection port in fluid communication with the mixing chamber, and
a first and a second cannula in fluid communication with the mixing chamber, and
an opening at a proximal end of the hollow body,
a first and a second cartridge each independently including:
a cartridge barrel body having:
a plunger slidably engaged with the cartridge barrel body, the plunger having a plunger shaft proximate the plunger, and
a seal at a distal end of the cartridge barrel body,
wherein the first and the second cartridges are each received within the elongated hollow body such that the seals of the first and second cartridges are adjacent the first and the second cannulas, and
an end cap connectable to the proximal end of the plunger shaft of each of the
first and the second cartridges;
providing a first material within the first cartridge;
providing a second material within the second cartridge; and
dispensing the first and the second materials within the dispenser.

21. The method of claim 20, wherein in the dispensing step, the first and the second materials are dispensed concurrently or sequentially.
